(19) United States
(12) Patent Application Publication
(10) Pub. No.: US 2026/0295017 A1
(43) Pub. Date: Oct. 1, 2026

(54) USE OF MIMETIC PEPTIDE CONTAINING FIBRINOGEN RGD MOTIF IN PREPARATION OF MEDICATIONS FOR PREVENTING AND/OR TREATING PARKINSON'S DISEASE

(71) Applicant: ZhuJiang Hospital of Southern Medical University, Guangzhou (CN)

(72) Inventor: Qing WANG, Guangzhou (CN)

(73) Assignee: ZhuJiang Hospital of Southern Medical University, Guangzhou (CN)

(21) Appl. No.: 19/572,918

(22) Filed: Mar. 20, 2026

(30) Foreign Application Priority Data
Apr. 1, 2025 (CN) 202510400214.6

(51) Int. Cl. A61K 38/36; A61P 25/16

(52) U.S. Cl. CPC A61K 38/363; A61P 25/16

(57) ABSTRACT

This disclosure belongs to the technical field of biomedicine, and discloses use of a mimetic peptide containing a fibrinogen RGD motif in preparation of medications for preventing and/or treating Parkinson's disease. A sequence of the mimetic peptide containing the fibrinogen RGD motif of this disclosure is: Gly-Arg-Gly-Asp-Ser-Pro-Leu-Ala-Pro-Ser-Cys. The applicant of this disclosure has found through research that there is abnormal accumulation of FG in substantia nigra pars compacta of PD mice, and that excessively accumulated FG in the brain may promote abnormal aggregation of α-syn in dopaminergic neurons under mediation of αvβ3 integrin receptor. The mimetic peptide provided by this disclosure can effectively inhibit the abnormal aggregation of α-syn in the dopaminergic neurons and death of the dopaminergic neurons caused by the abnormally accumulated FG in the brain, improve a motion ability of MPTP-modeled PD mice, and provide a new target and idea for PD treatment.

### CROSS-REFERENCE TO RELATED APPLICATIONS

The application claims priority to Chinese patent application No. 202510400214.6, filed on Apr. 1, 2025, the entire contents of which are incorporated herein by reference.

### TECHNICAL FIELD

This disclosure belongs to the technical field of biomedicine, and particularly relates to use of a mimetic peptide containing a fibrinogen RGD motif in preparation of medications for preventing and/or treating Parkinson's disease.

### BACKGROUND

Parkinson's disease (PD), as the second most common neurodegenerative disease, has its main pathological hallmarks as the destruction of dopaminergic neurons in the substantia nigra and the formation of Lewy bodies containing α-synuclein (α-syn). Currently, there are no effective radical cure regimens and therapeutic medications for PD. Therefore, it is of great clinical significance to provide more potential therapeutic targets and treatment strategies for PD through research on pathological mechanisms of PD.

In PD, a pathological mechanism of abnormal aggregation of α-syn is very complex. Research has shown that destruction of a blood-brain barrier in PD is also closely related to the abnormal aggregation of α-syn. The applicant of this disclosure has found through research that the destruction of the blood-brain barrier in PD mice can lead to a large amount of plasma-derived fibrinogen (FG) entering the brain tissue, and the aggregation of FG in the brain can lead to abnormal aggregation of α-syn and damage to dopaminergic neurons in substantia nigra pars compacta. FG is a heterodimeric glycoprotein with a molecular weight of about 340 kDa, and is synthesized by liver cells. Under normal physiological conditions, FG is mainly present in plasma and participates in the blood coagulation process in response to injury in blood vessels. In 2021, Silva et al. reported in the journal of Science that the interactions between FG and cells mainly relied on integrin receptors. Integrin receptors are the main receptors for FG to bind to platelets, endothelial cells or macrophages in peripheral blood. They are widely present in various types of cells and act as an important bridge for interaction between the extracellular matrix and cells. Further research by the applicant of this disclosure has found that FG can promote the abnormal aggregation of α-syn in neurons and the increase of phosphorylated α-syn through the mediation of neuronal αvβ3 integrin receptors, and lead to mitochondrial damage in dopaminergic neurons, ultimately inducing the death of dopaminergic neurons; inhibiting the binding of FG to αvβ3 integrin receptors can effectively delay the pathological changes of PD.

GRGDSPLAPSC is a peptide containing arginine-glycine-aspartic acid (RGD). GRGDSPLAPSC is a competitive and reversible inhibitory peptide that can inhibit integrin-fibronectin binding. Current research has found that GRGDSPLAPSC can be used to research the role of integrins in bone formation and resorption. To date, no relevant research has been reported regarding the utilization of small-molecule peptides related to an RGD motif for the preparation of medications for treating PD.

### SUMMARY

To address the drawbacks and deficiencies in the prior art mentioned above, a primary objective of this disclosure is to provide use of a mimetic peptide containing a fibrinogen RGD motif in preparation of medications for preventing and/or treating Parkinson's disease.

The objective of this disclosure is achieved through the following technical solutions:

A mimetic peptide containing a fibrinogen RGD motif, wherein a sequence of the mimetic peptide is: Gly-Arg-Gly-Asp-Ser-Pro-Leu-Ala-Pro-Ser-Cys (GRGDSPLAPSC).

Among them, glycine (Gly, G), arginine (Arg, R), aspartic acid (Asp, D), serine (Ser, S), proline (Pro, P), leucine (Leu, L), alanine (Ala, A), proline (Pro, P), and cysteine(Cys, C).

Further, a protein molecular weight of the peptide is 1059.1.

The mimetic peptide described in this disclosure can be synthesized by Fmoc solid-phase synthesis, and its purity is identified by multi-effect liquid chromatography analysis.

Specifically, the following steps may be included:
1. Resin swelling

Place resin (2-chlorotrityl chloride resin) into a reaction tube, add DMF (15 mL/g), and shake for 60 min.
2. Loading of a first amino acid

Perform suction filtration to remove a solvent through a fritted disc; add a 3-fold molar excess of Fmoc-protected amino acid (the first amino acid at the C-terminal), then add a 10-fold molar excess of DIEA, and finally add DMF for dissolution; shake for 30 min; cap with methanol for 30 min.
3. Deprotection

Remove DMF, add a solution of 20% piperidine in DMF (15 mL/g), 5 min, remove it, and add again a solution of 20% piperidine in DMF (15 mL/g), 15 min.
4. Detection

Suck away a piperidine solution, take about a dozen pieces of resin, wash three times with ethanol, add 2-3 drops of Kaiser reagent, and heat at 105° C.-110° C. for 5 min, where a dark blue color indicates a positive reaction.
5. Washing

Wash twice with DMF (10 mL/g), twice with methanol (10 mL/g), and twice with

DMF (10 mL/g).
6. Condensation

Add a 3-fold molar excess of Fmoc-protected amino acid and a 3-fold molar excess of HBTU, then add a 10-fold molar excess of DIEA, finally add DMF for dissolution, and shake for 45 min.
7. Detection

Take about a dozen pieces of resin, wash three times with ethanol, add 2-3 drops of Kaiser reagent, and heat at 105° C.-110° C. for 5 min, where a colorless result indicates a negative reaction.
8. Washing

Wash once with DMF (10 mL/g), twice with methanol (10 mL/g), and twice with DMF (10 mL/g).
9. Repeat operations of 3-8, and perform loading from right to left in sequence until the Fmoc protecting group of the last amino acid is removed.
10. Washing and suction drying of resin according to the following methods

Wash twice with DMF (10 mL/g), three times with DCM (10 mL/g), and four times with methanol (10 mL/g), and perform suction drying for 10 min.
11. Cleavage

Prepare a cleavage solution (10 mL/g): 95% of TFA; 2% of water; 2% of EDT; 1% of TIS. Cleavage time: 180 min.
12. Blow drying and washing

Blow a lysate as dry as possible with nitrogen, precipitate with ether, remove the supernatant by centrifugation, wash the precipitate six times with ether, and then evaporate to dryness at room temperature.
13. Purification and preparation
   (1) Take a small amount of a crude product and dissolve it in H₂°/ACN.
   (2) Take a small amount of sample and analyze it on the HPLC analysis instrument to determine an appearance time corresponding to a target peak.
   (3) Use a C18 reversed-phase chromatography preparation system: Wavelength: 220 nm; Flow Rate: 15 mL/min; Inj. Vol: 20 mL; Column Temp: 25° C.; Buffer A: 0.1% TFA in water; Buffer B: 0.1% TFA in Acetonitrile, for collecting a target peak solution.
   (4) Take a small amount of the target peak solution with a 1.5 mL centrifuge tube for mass spectrometry confirmation and purity testing.
14. Lyophilize a qualified target peak solution.
15. Identification: Respectively take a small amount of finished peptide for molecular weight identification by MS and purity identification by HPLC analysis.
16. Seal and package a powdered peptide and store at −20° C.

This disclosure further provides use of a mimetic peptide containing a fibrinogen RGD motif in preparation of medications for preventing and/or treating Parkinson's disease mentioned above.

In the technical solutions of this disclosure, the medications, whether identical or different, respectively comprise a therapeutically effective amount of mimetic peptide containing the fibrinogen RGD motif.

In the technical solutions of this disclosure, the medications, whether identical or different, can be respectively prepared into various medicinal dosage forms by conventional methods, and these dosage forms comprise oral dosage forms such as tablets, sugar-coated tablets, film-coated tablets, enteric-coated tablets, capsules, hard capsules, soft capsules, oral solutions, buccal tablets, granules, soluble granules, pills, pellets, suspensions, medicinal liquors, tinctures, drops, as well as injection solutions and other dosage forms other than oral dosage forms, such as injections.

In the technical solutions of this disclosure, the medications, whether identical or different, may further respectively contain one or more pharmaceutically acceptable carriers or excipients.

Further, the carriers or excipients may comprise diluents, wetting agents, binders, surfactants, humectants, adsorbent carriers, lubricants, fillers, disintegrants, preservatives, etc.

The applicant of this disclosure has found through research that there is abnormal accumulation of FG in substantia nigra pars compacta of PD mice, and that excessively accumulated FG in the brain may promote abnormal aggregation of α-syn of dopaminergic neurons under mediation of αvβ3 integrin receptor. The mimetic peptide provided by this disclosure can effectively inhibit the abnormal aggregation of α-syn of the dopaminergic neurons and death of the dopaminergic neurons caused by the abnormally accumulated FG in the brain, improve a motion ability of MPTP-modeled PD mice, and provide a new target and idea for PD treatment.

### BRIEF DESCRIPTION OF DRAWINGS

To illustrate the technical solutions of implementations of this disclosure more clearly, drawings required to be used in the implementations are briefly introduced below. It should be understood that the following drawings show merely certain implementations of this disclosure and are therefore not to be considered limited in scope, and for those of ordinary skill in the art, other related drawings can also be obtained without exerting creative efforts according to these drawings.

FIG. 1 is a diagram showing an effect of a mimetic peptide of this disclosure in reversing up-regulation of α-syn multimer expression induced by FG intervention.

FIG. 2 is a diagram showing effects of Batroxobin in reducing levels of α-syn multimers and phosphorylation in substantia nigra of MPTP-modeled PD mice.

FIG. 3 is a diagram showing that αvβ3 integrin receptor inhibitor reduces pathological abnormal aggregation of α-syn in SH-SY5Y cells induced by FG.

FIG. 4 is a diagram showing an effect of the mimetic peptide of this disclosure in reducing the loss of dopaminergic neurons in MPTP-modeled PD mice.

FIG. 5 is a diagram showing an effect of the mimetic peptide of this disclosure in reversing dyskinesia of MPTP-modeled PD mice.

### DETAILED DESCRIPTION OF THE EMBODIMENTS

This disclosure will be described in further detail below with reference to implementations, but the embodiments of this disclosure are not limited thereto. The materials involved in the following implementations can be obtained from commercial sources unless otherwise specified. The methods described are conventional methods unless otherwise specified.

According to the mimetic peptide of this disclosure, through computer-aided medication design (cadd), up to 20,000 peptide medications or other known small-molecule medications are screened from existing peptide libraries according to an RGD peptide segment of FG, then mimetic peptides containing RGD sites of fibrinogen (FG) α-chains are designed by matching an αvβ3 integrin target, and finally, the optimal mimic peptide capable of binding to the target is screened according to binding energy parameters, where a peptide sequence is GRGDSPLAPSC.

The mimetic peptide used in this implementation was entrusted to ChinaPeptides Co., Ltd. for synthesis by Fmoc solid-phase synthesis, with a purity of the peptide of more than 95%. GRGDSPLAPSC can competitively inhibit the binding of FG to the neuronal αvβ3 integrin receptor by mimicking the RGD site in FG.

Source of reagents: αvβ3 integrin receptor inhibitor (cyclo (RGDyK)): Supplier MCE article number: HY-100563A; αvβ5 integrin receptor inhibitor (αvβ5 integrin-IN-1): Supplier MCE article number: HY-145363; α5β1 integrin receptor inhibitor (ATN-161): Supplier MCE article number: HY-13535.

### Implementation 1

Human neuroblastoma cells (SH-SY5Y cell line) expressing tyrosine hydroxylase (TH) were selected for in vitro culture. After a high-glucose DMEM medium containing 10% fetal bovine serum (FBS) and 1% penicillin-streptomycin was replaced for resuscitated cells, cell solution replacement could be performed every 1-2 days under normal cell growth, cell growth vigor and status should be observed every day, and attention should be paid to aseptic operation to avoid cell contamination.

When the cells grew to cover about 70%-90% of a bottom surface, cell passage was performed. The old cell culture medium was aspirated with a pipette tip, and an appropriate amount of sterile PBS buffer was added to clean the bottom surface of a culture flask. After the PBS buffer was completely aspirated, as SH-SY5Y served as adherent cells, about 1 mL of trypsin digestion solution required to be added for passage, and digestion was carried out at room temperature for 1-2 min. When most cells were observed to round up under a microscope, 1 mL of DMEM-containing medium was added to terminate digestion. One day before treatment with fibrinogen (FG), SH-SY5Y cells were counted with a cell counter. If the cells grew well without contamination, solution replacement for the cells was performed with serum-free and antibiotic-free DMEM culture solution for the cells in each well.

Then, 400 μg/mL of human serum-derived FG was added to the culture medium, and 400 μg/mL of GRGDSPLAPSC was added to the GRGDSPLAPSC-treated groups respectively. After addition, the culture solution was shaken thoroughly, and the cells were further incubated in a cell incubator for 48 hours. The cells were observed every 12 hours for normal growth and morphology, and the cells were collected after 48 hours for tests.

After the intervention time (48 hours) was ended, the cell culture medium was discarded, and the cells were washed 3 times with PBS buffer. All the cells were then scraped off with a cell scraper, and the scraped/pipetted cells were transferred to 1.5 mL EP tubes. The cell precipitates were retained after centrifugation at 3000 r/min for 10 min at 4° C. A certain amount of PBS buffer was then added, and centrifugation was performed again at 3000 r/min for 10 min at 4° C. Afterwards, the precipitates were retained, and a total cellular protein lysate mixture was added. 100-200 μL of the prepared total protein lysate was added to each tube of cells, repeatedly pipetted up and down, shaken to mix well, and the samples were placed on ice for 30-60 min. Then centrifugation was carried out at 12000 r/min for 15 min at 4° C., and the supernatant was taken into new pre-cooled 1.5 mL EP tubes. These were the extracted total protein samples. Subsequently, Western Blot tests were performed using the total protein samples to detect the protein expression level of total α-syn.

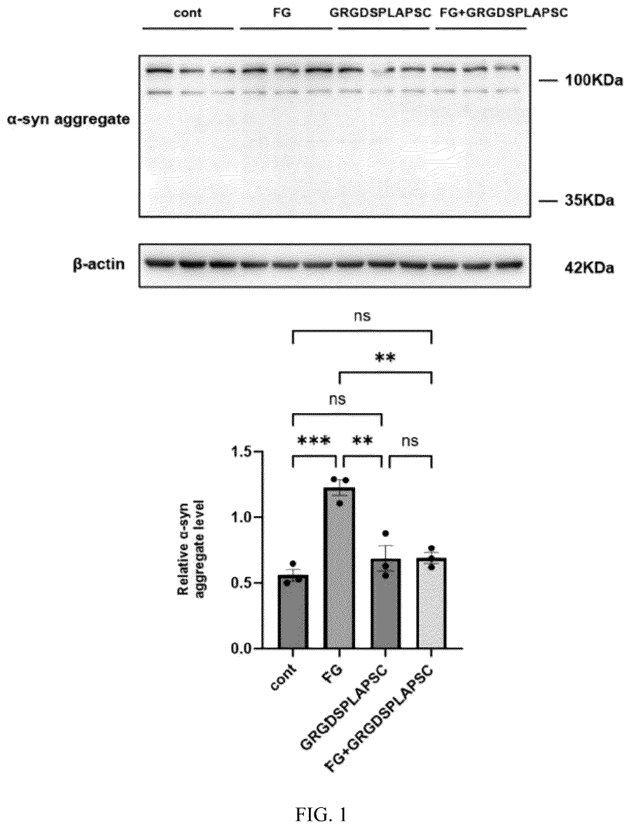

The experimental results showed that FG intervention increased the expression of α-syn multimers, but treatment of cells with 400 μg/mL GRGDSPLAPSC could significantly reverse this trend, as shown in FIG. 1.

### Implementation 2

40 healthy adult C57BL/6 male mice weighing about 25 g were taken and randomly divided into four groups. One group of mice was injected with normal saline; one group of mice was subjected to intraperitoneal injection of 30 mg/mL of 1-methyl-4-phenyl-1,2,3,6-tetra-hydropyridine hydrochloride (MPTP) once daily for modeling for 5 consecutive days. One MPTP group was injected with 30 BU/kg/d of Batroxobin during modeling to promote the reduction of FG in the whole blood (Batroxobin was injected after the MPTP injection on day 5), and simultaneously one normal saline group was injected with the same dose of Batroxobin as a control. The mice were sacrificed 16 days after the modeling was completed, and the brain tissues of the substantia nigra pars compacta of the mice were taken for grinding and centrifugation. Total protein was extracted for Western Blot tests to detect the protein expression levels of total α-syn and phosphorylated α-syn.

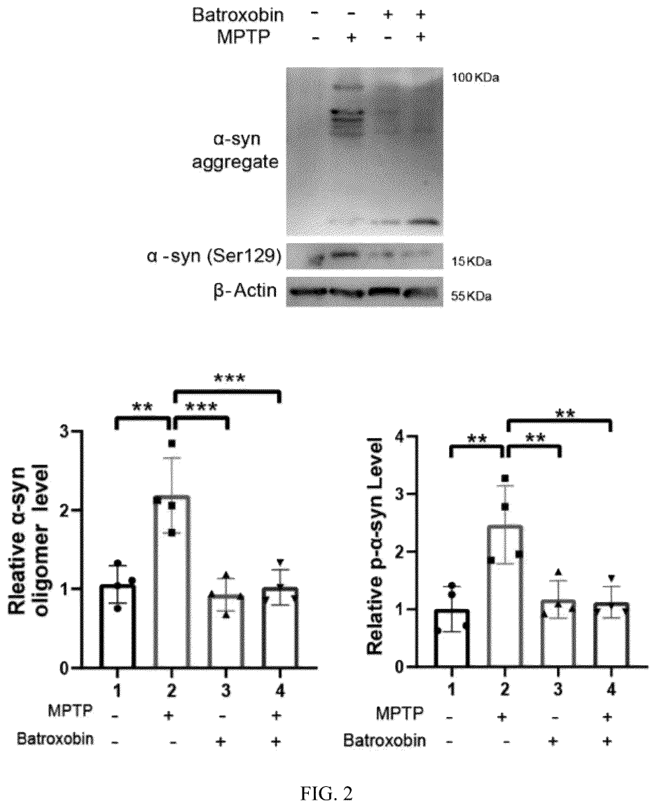

The experimental results showed that Batroxobin could significantly reduce the level of pathological α-syn protein in the substantia nigra of the MPTP-modeled PD mice after reducing the level of FG in whole blood, suggesting that FG may play a role in promoting the abnormal aggregation of α-syn in PD mice, and reducing the level of FG can effectively reverse the aggregation process of α-syn, as shown in FIG. 2.

### Implementation 3

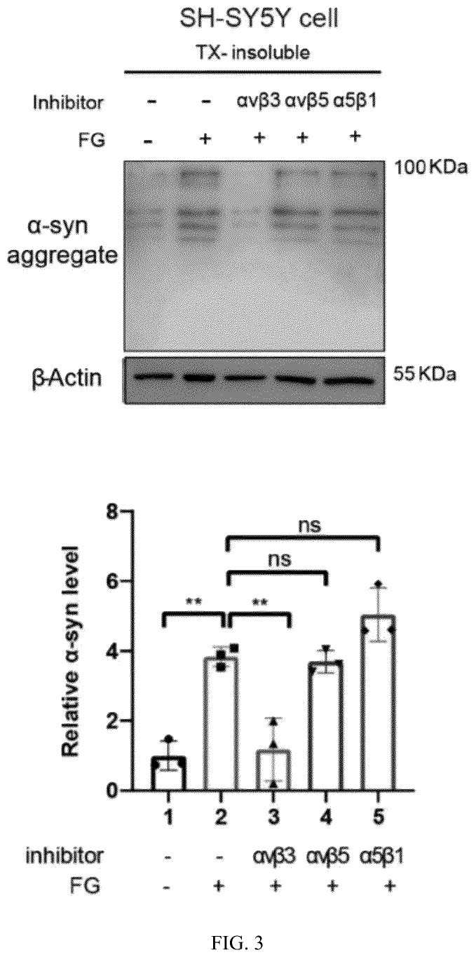

The SH-SY5Y cell line was passaged into 6-well plates, 2 mL of DMEM culture solution was added to each well, and the cells were randomly divided into 5 groups. For the FG-treated group, 400 μg/mL of human serum-derived FG was added to the culture medium, and for the normal control group, an equal amount of PBS buffer was added. On the basis of FG treatment, the remaining three groups were added with corresponding concentrations of αvβ3 integrin receptor inhibitor, αvβ5 integrin receptor inhibitor, and α5β1 integrin receptor inhibitor respectively according to the manufacturer's instructions. After 48 hours, the cells were collected, total protein was extracted, and Western Blot tests were performed to detect the protein expression level of total α-syn multimers. The experimental results suggested that the αvβ3 integrin receptor inhibitor significantly reduced the pathological abnormal aggregation of α-syn induced by FG, suggesting that FG promotes α-syn aggregation through the mediation of the αvβ3 integrin receptor, as shown in FIG. 3.

### Implementation 4

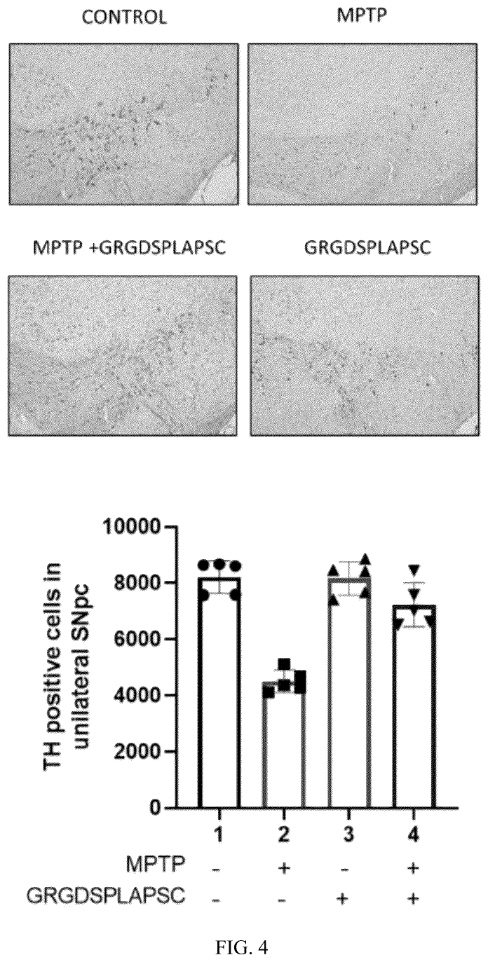

40 healthy adult C57BL/6 male mice weighing about 25 g were taken and randomly divided into four groups. One group of mice was injected with normal saline; one group of mice was subjected to intraperitoneal injection of 30 mg/mL of 1-methyl-4-phenyl-1,2,3,6-tetra-hydropyridine hydrochloride (MPTP) once daily for modeling for 5 consecutive days; one group of mice was subjected to MPTP modeling and GRGDSPLAPSC (5 days before MPTP modeling, GRGDSPLAPSC was diluted to 1.5 mg/mL with ACSF (artificial cerebrospinal fluid, commonly available commercially), and stereotaxic injection of 2 μL is performed on the substantia nigra pars compacta); meanwhile, one normal saline group was injected with the same dose of GRGDSPLAPSC as a control. The mice were sacrificed 16 days after the MPTP modeling, and the whole brains were taken, fixed, dehydrated, and then sectioned to obtain substantia nigra brain slices. The number of dopaminergic neurons (tyrosine hydroxylase TH-positive neurons) in the substantia nigra of the mice was detected by immunohistochemistry. The experimental results showed that GRGDSPLAPSC can significantly reduce the loss of dopaminergic neurons in MPTP-modeled PD mice, as shown in FIG. 4.

### Implementation 5

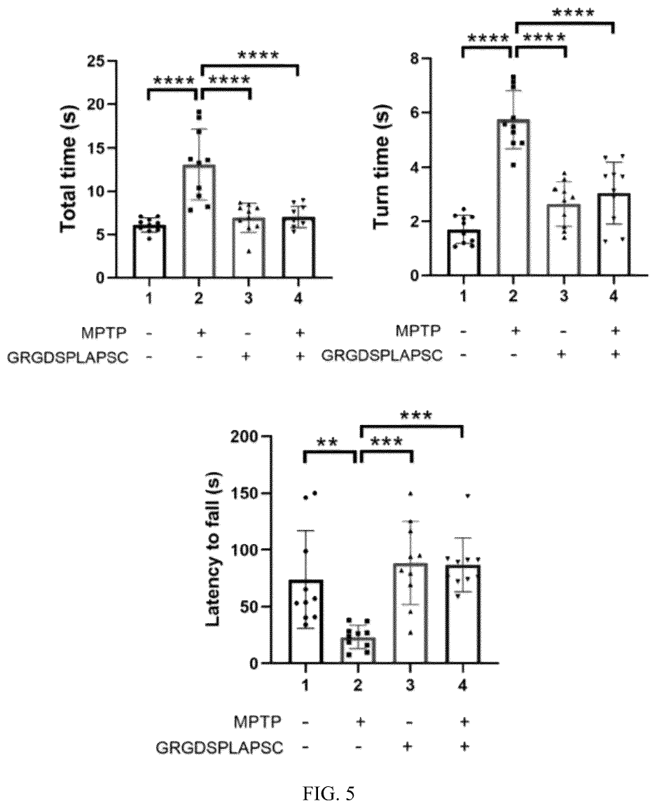

In Implementation 4, the mice were subjected to behavioral detection 3 days before sacrifice, including: (1) rotarod test: the mice were trained for 3 days before the test. The mice were placed on a fixed rod, and the rotation speed was gradually increased to 5 rpm to train the mice to rotate with the rod. The training was conducted 5 times per day for 3 consecutive days at a speed of 5 rpm (maximum of 10 rotations per training session), and the mice were required to reach the optimal status within 180 seconds. After 3 days of habituation training, the test was initiated. Each mouse was placed in the center of a stationary cylinder with its body axis perpendicular to the cylinder axis, and the rotation speed was gradually increased to 5 rpm. The time that the mouse remained on the cylinder without falling was recorded (any time exceeding 180 seconds was recorded as 180 seconds). Each mouse was tested 3 times, each with an interval of 30 min, and the average value was taken; (2) Pole climbing test: the wooden pole had a width of 0.6 cm, a height of 50 cm, and a rough surface. All mice were subjected to habituation training 1 day before the test. During the test, each mouse was placed on the top of the wooden pole with its head facing upwards. The time to turn around and face downward and the time to climb from the top of the pole to the ground (all four limbs were required to be completely on the ground) were recorded. The test was conducted for 5 consecutive times, and the best performance was taken as the final result. The results were shown in FIG. 5. From left to right in the figure, the total time of the pole climbing test, the turn time, and the time of rotarod test were shown in sequence. The experimental results showed that GRGDSPLAPSC can significantly reverse the dyskinesia of the MPTP-modeled PD mice.

The above implementations are preferred embodiments of this disclosure, but the embodiments of this disclosure are not limited by the above implementations. Any other changes, modifications, substitutions, combinations, and simplifications that do not deviate from the spirit and principles of this disclosure should be equivalent replacement methods, and are all included in the scope of protection of this disclosure.

What is claimed is:

1. Use of a mimetic peptide containing a fibrinogen RGD motif in preparation of medications for preventing and/or treating Parkinson's disease.

2. The use according to claim 1, wherein a sequence of the mimetic peptide is: Gly-Arg-Gly-Asp-Ser-Pro-Leu-Ala-Pro-Ser-Cys.

3. The use according to claim 1, wherein the medications comprise a therapeutically effective amount of the mimetic peptide containing the fibrinogen RGD motif.

4. The use according to claim 1, wherein the medications are prepared into various medicinal dosage forms by conventional methods, and these dosage forms comprise oral dosage forms of tablets, capsules, oral solutions, buccal tablets, granules, soluble granules, pills, pellets, suspensions, medicinal liquors, tinctures and drops, injection solutions, and intranasal dosage forms including nasal sprays, nasal drops, nasal solutions, nasal suspensions, nasal powders, and nasal gels.

5. The use according to claim 1, wherein the medications are prepared into various medicinal dosage forms by conventional methods, and these dosage forms comprise oral dosage forms of sugar-coated tablets, film-coated tablets, enteric-coated tablets, hard capsules, soft capsules, oral solutions, buccal tablets, granules, soluble granules, pills, pellets, suspensions, medicinal liquors, tinctures and drops, injection solutions, and intranasal dosage forms including nasal sprays, nasal drops, nasal solutions, nasal suspensions, nasal powders, and nasal gels.

6. The use according to claim 1, wherein the medications further comprise one or more pharmaceutically acceptable carriers or excipients.

7. The use according to claim 1, wherein the carriers or excipients comprise at least one of diluents, wetting agents, binders, surfactants, humectants, adsorbent carriers, lubricants, fillers, disintegrants, and preservatives.
